(19) United States
(12) Patent Application Publication
(10) Pub. No.: US 2004/0062719 A1
(43) Pub. Date: Apr. 1, 2004

(54) Particulate inhalation carrier

(76) Inventors: Graham Buckton, London (GB); Dima Al-Hadithi, London (GB); Stephen Brocchini, London (GB)

Correspondence Address: Dickstein Shapiro Morin & Oshinsky, 41st Floor, 1177 Avenue of the Americas, New York, NY, 10036-2714, US

(21) Appl. No.: 10/433,435

(22) PCT Filed: Dec. 10, 2001
(86) PCT No.: PCT/GB01/05436

(30) Foreign Application Priority Data
Dec. 8, 2000 (GB) 0030074.9

(51) Int. Cl. A61L 9/04; A61K 38/00; A61K 9/14; A61K 31/7012; A61K 38/28

(52) U.S. Cl. 424/46; 514/53; 514/2; 514/171; 514/179; 514/3

(57) ABSTRACT

The present invention provides a particulate substrate suitable for carrying a drug for delivery, comprising a substantially crystalline core and a surface coating, wherein the particulate substrate has a proportion of amorphous character of 2% of greater by weight of particulate substrate, and a process for the production of carrier particles comprising the steps of: a) mixing crystalline particles having an average diameter greater than 10 μm with at least partially amorphous particles having average diameters less than 10 μm; b) exposing the mixture to conditions capable of inducing crystallization of the amorphous particles for a predetermined period in order that partial crystallization takes place.

### FILED OF INVENTION

The present invention is concerned with the field of drug delivery via inhalation.

### BACKGROUND OF INVENTION

The delivery of drugs to the lung is becoming increasingly common, both to treat-diseases of the lung, such as asthma, and as a means of systemic delivery. Dry powder inhalers are becoming ever more popular, especially since the removal of CFC based metered dose inhalers as a means of therapy. Dry powder inhalers can consist of the drug alone, in micronised form so as to allow inhalation into the deep lung, or of the micronised drug mixed with a larger carrier particle. Formulations based on drug only suffer from difficulties in dosing of small quantities of fine particles, as these tend to have very poor flow properties (Byron, P. R., 1986. Some future perspectives for unit dose inhalation aerosols. Drug Dev. Ind. Pharm., 12, 993-1015.). This is because it is generally necessary to have particles of 1-5 um in order to achieve deep lung deposition (Neumann, S. P. and Clarke, S. W., 1983, Therapeutic Aerosols, I. Physical and practical considerations. Thorax 38, 881-886.). For drugs with carrier particles, it is necessary to first mix the fine drug so that it attaches to the large carrier, thus enabling flow and accurate dosing. Subsequently, during breath actuation of an inhaler, it is necessary for the drug particles to detach from the carrier surface, allowing the fine particles to be inhaled and the carrier to be deposited on the throat and passed down the gasto-intestinal tract.

Most dry powder inhaler (DPI) devices that have a carrier particle utilise lactose, possibly with a mixture of more than one size fraction to give optimum results (Zeng et al (Zeng, X. M., Pandhal, K, H., Martin, G. P., Int. J. Pharmaceutics, vo. 197, 41-52, 2000). The influence of lactose carrier on the content homogeneity and dispersibility of beclomethasone dipropionate from dry powder aerosols have shown that addition of fines, and the order in which coarse lactose, fine lactose and drug are mixed will influence the drug deposition. A few alternatives to lactose have been considered, for example the use of a ternary component deposited on the lactose surface in order to fill void spaces has been proposed (Ganderton, D., Kassem, N. M., 1992. Dry powder inhaler. In Ganderton, D., (ed) Advances in Pharmaceutical Sciences. Academic Press, London pp 165-191).

Leucine has been suggested as a suitable ternary component and has been found to give favourable deposition compared to lactose alone (Staniforth, J. N., 1996. Improvement in dry powder inhaler performance: surface passivation effects. Proc. Drug Delivery Lungs (London) VII, 86-89). Kawashima et al (Kawashima, Y., Serigano, T., Hino, T., Yamamoto, H., Takeuchi, H., 1998a; Design of inhalation dry powder of pranlukast hydrate to improve dispersibility by the surface modification with anhydrous silic acid (AEROSIL 200). Int. J. Pharm., 173, 243-251) utilised colloidal silica as a ternary component in order to make the drug surface have a more hydrophilic nature (particles were either mixed with colloidal silica or lyophilised or spray dried).

Tee et al (Tee, S. K., Marriott, C., Zeng, X. M. and Martin, G. P., 2000, The use of different sugars as fine and coarse carriers for aerosolised salbutamol sulphate. Int. J. Pharm., 208,111-123.) have considered the use of different carbohydrates (mannitol and sorbitol as well as lactose) and they concluded that the carriers were all ineffective unless fine particles were added (for the delivery of salbutamol sulphate). Furthermore, Tee et al (2000) state that the nature of the small particle is not significant, but rather the presence of small particles as an addition to the large carrier particles are the key to achieving effective drug release.

Kawashima et al (Kawashima, Y., Serigano, T., Hino, T., Yamamoto, H., Takeuchi, H., 1998b. Effect of surface morphology of carrier lactose on dry powder inhalation property of pranlukast hydrate. Int. J. Pharm., 172, 179-188.) utilised different physical forms of lactose as carrier particles and concluded that the surface roughness was key to drug release from the carrier, it was concluded that smooth surfaces allowed more drug to be released. Changes in surface smoothness and elongation ratio of lactose crystals were found to improve deposition of salbutamol sulphate by Zeng et al (Zeng, X. M., Martin G. P., Marriott, C., Pritchard, J., 2000b. The influence of carrier morphology on drug delivery by dry powder inhalers. Int. J. Pharm., 200, 93-106.).

WO01/05429 describes a method of preparation of carriers for inhalation powders, consisting of particles with a smooth surface. The method affords smooth particles starting from an industrial powder consisting of rough particles, without substantially altering their average size and their geometry. The carrier is prepared using a high-speed mixer-granulator, an apparatus designed and normally used for agglomerating solid particles and not for smoothing them individually.

WO96/23485 discloses a powder for use in a dry powder inhaler, including active particles and carrier particles for carrying the active particles.

The powder further includes additive material on the surfaces of the carrier particles to promote the release of the active particles from the carrier particles on actuation of the inhaler. The additive materials are identified as amino acids, lecithin and magnesium stearate.

### SUMMARY OF INVENTION

According to a first aspect of the present invention, there is provided a particulate substrate suitable for carrying a drug for delivery, comprising a substantially crystalline core and a surface coating, wherein the particulate substrate has a proportion of amorphous character of 2% or greater by weight of particulate substrate.

In a second aspect, the present invention provides a process for the production of carrier particles comprising the steps of:

a) mixing substantially crystalline particles having an average diameter greater than 10 μm with at least partially amorphous particles having average diameters less than 10 μm;

b) exposing the mixture to conditions capable of inducing crystallization of the amorphous particles for a predetermined period in order that partial crystallization takes place.

### DESCRIPTION OF THE PREF ERRED EMBODIMENTS

Generally, the particulate substrate of the present invention comprises a substantially crystalline core upon which a coating of particles or fluid is applied and which forms a surface having an at least partially amorphous structure. Preferably, the surface of the particulate substrate itself has sufficient amorphous character such that the overall amorphous content of the particulate substrate is 2% or greater, even though the core is substantially crystalline. The surface is therefore defined as the coating of particles or fluid regardless of what physical changes are effected to said coating. The particle including the core and surface coating will hereinafter be referred to as a carrier particle. The surface coating need not be continuous and, particularly in the case where a coating of particulate material is applied to the core, the coating may be discontinuous. The proportion of the surface of the crystalline core which is covered by the amorphous coating may be in the range of 0.1% to 95%, preferably 0.5% to 80%.

Although the crystalline state is generally the most stable form, the intention of the present invention is to produce a particle retaining a degree of amorphous character.

The carrier particle may be uniform in dimensions, but non-uniform carrier particles may be produced. The largest diameter of the carrier particles is generally in the range of 10 μm to 500 μm, preferably 20 μm to 100 μm, most preferably 45 μm to 90 μm.

The carrier particle should not have a diameter of less than 10 μm otherwise inhalation of the carrier particle into the deep lung may occur. Although this is not harmful, it is undesirable.

The carrier particle is generally formed from a pharmaceutically inert material. For example, saccharides are commonly used in the field of inhalation drug delivery and these are preferred materials for the formation of the carrier particles of the present invention.

Preferably the core material and the surface coating are formed from the same material. However, this is not necessary and a heterogenous particle may be produced with a surface coating formed from a different material than the particle core.

Preferably the particle core and the surface coating are individually selected from the group consisting of lactose, sucrose, glucose, galactose, fructose, trehalose, raffinose and mixtures thereof. In a particularly preferred embodiment, lactose is used to form both the particle core and the surface coating.

The carrier particle core is substantially crystalline. Preferably, greater than 90%, more preferably greater than 98%, most preferably, greater than 99% of the core has a crystalline structure.

The surface coating has an at least partially amorphous structure. In particular, the surface coating should have a relatively greater amorphous character than the core of the particle. Preferably, the surface coating has a proportion of amorphous character such that the total amorphous content of the carrier particle is in the range of 2 to 80%, more preferably 3 to 20%, more preferably 3.5 to 8%, most preferably about 4 to 7% by weight of the carrier particle. In a particularly preferred embodiment, the surface coating has a proportion of amorphous character of about 6% by weight of the carrier particle.

Preferably, the product of the present invention is substantially absent of free fine particles. The majority of the fine particles are incorporated into the carrier particles.

With regard to the process according to the second aspect of the present invention, the substantially crystalline particles having an average diameter greater than 10 μm (hereinafter referred to as coarse particles) are generally sieved or otherwise separated from fines or large particles not suitable for delivery by inhalation. Generally, the course particles have a diameter in the range of 10 μm to 500 μm, preferably 20 μm to 100 μm, most preferably 45 μm to 90 μm.

Coarse particles of lactose are preferably prepared by sieving commercially available pharmaceutical lactose, for between 1 and 60 minutes, over a mesh to remove substantially all of the fines.

The at least partially amorphous particles (hereinafter referred to as fine particles) generally have a diameter in the range of about 0.1 μm to about 10 μm, preferably about 1 μm to about 8 μm.

In a preferred embodiment the at least partially amorphous particles are prepared by spray drying of an aqueous solution of lactose. These particles preferably have an amorphous character of greater than 10% by weight of the spray dried product. More preferably, these particles have an amorphous character of greater than 50%, most preferably between 90 and 100% at the point of spray dried production. Preferably, the fine particles also have a similar amorphous content at the point of preparation of the carrier particles. Alternatively, the amorphous particles may be produced by a process of freeze drying or precipitation.

After preparation of the amorphous fine particles, the coarse particles are mixed with the fine particles. Generally mixing can take place by any suitable means, for example a Turbula type mixer. The mixing time is in the range from 5 seconds to 24 hours, more preferably 1 minute to 1 hour, most preferably about 5 to 30 minutes.

The proportion of coarse particles to fine particles in the mix is generally in the ratio range of from 20:1 to 5:1, preferably 12:1 to 7:1 by weight of the mixture. In a particularly preferred embodiment, the coarse particles to fine particles ratio is about 9:1 by weight of the mixture.

At this point during the production of the carrier particles, the coarse particles generally have a plurality of fine particles deposited thereon. Generally, fine particles loosely adhere to the coarse particle surface. The carrier particle may then be exposed to process conditions (hereafter referred to as conditioning) capable of causing at least some of the amorphous particle structure to change to a crystalline structure. Preferably, the conditioning involves exposure of the intermediate carrier particles (not yet conditioned by crystallisation) to a humid environment. This may be achieved by placing the intermediate carrier particles in a receptacle adapted for throughput of fluid, preferably gas. The gas preferably contains water with a humidity level suitable for causing crystallisation of the amorphous fine particles. Alternatively, the gas may contain organic vapour capable of causing conditioning of the fine particles. Such an organic vapour is preferably an alcohol, most preferably ethanol.

In the preferred embodiment, the gas comprises water vapour. This is then pumped through or over the carrier particle mixture for a predetermined period. The exposure to the water vapour is generally for a period in the range of 10 seconds to 48 hours, preferably 10 minutes to 6 hours, more preferably 30 minutes to 5 hours, most preferably 1 hour to 3 hours.

Elevated temperature may also be used to induce crystallization of the fine particles. Preferably a temperature is used in the range of 5° C. to 200° C., more preferably 10° C. to 80° C., most preferably 15° C. to 50° C. Preferably this is done in combination with some level of humidity.

The extent to which crystallization of the fine particles occurs in the apparatus can be controlled by changing the relative humidity, temperature, the exposure time, flow rate of gas, powder mass of substrate, volume of substrate or combinations thereof.

The relative humidity of the gas is preferably in the range of 1 to 100%, more preferably 30% to 80%, most preferably 40 to 60% relative humidity. In a particularly preferred embodiment, the relative humidity is 53% for a duration of approximately 2 hours. Typically, 5 to 10 grams of substrate are conditioned at a time, at a gas flow rate of 0.2 litres/minute.

The carrier particles may be sieved to remove large aggregates. Preferably sieving takes place after the drying step.

After exposure to the conditioning process, the surface coating generally has a proportion of amorphous structure in the range of 0.1% to 95%, preferably 0.5% to 80%, more preferably 2% to 65%, most preferably 40% to 60% by weight of the total surface coating. As shown in Table 3, the final amorphous content of the entire carrier particle may be measured. Thus, for any given amount of initially added fines (with a known amorphous content) to a given amount of course crystalline particles, the amorphous content of the coating can be calculated. For any given amount of initially added fines to a given amount of course particles, under specific humidification conditions, the final amorphous % contents (measurable) and a range of conditioning periods may be plotted to provide a graphical representation of the total amorphous content of the entire carrier particle, for any given conditioning period. Thus the conditioning period may be altered to provide a product having a desired amorphous content.

After conditioning, the carrier particles may be dried. Drying may take place either at elevated temperatures, by the use of a desiccant, at reduced pressure or a mixture thereof. Preferably, drying takes place for a period of between 1 hour and 5 days. In a particularly preferred embodiment, drying takes place for approximately 24 hours by passing dried air through or over the carrier particles.

The product of the present invention may be treated with a pharmaceutically active agent. The pharmaceutically active agent is preferably a drug, most preferably a drug which may be delivered by inhalation. Generally, the pharmaceutically active agent is deposited, preferably coated onto the surface of the carrier particle.

In a preferred embodiment, the pharmaceutically active agent is mixed with the carrier particles after the drying step.

The pharmaceutically active agent is preferably provided in micronised form.

The proportion of pharmaceutically active agent to carrier particles is generally in the range of 1:1 to 1:100, preferably 1:3 to 1:50 although any mixture may provided which allows delivery of a predetermined dosage of drug to the subject via inhalation of the carrier particles.

In a particularly preferred embodiment, between 1 and 5% w/w pharmaceutically active agent is mixed with the carrier particles. Blending may be effected by any means. For example, blending takes place in a Turbula mixer. Blending generally takes place for period of between 1 minute and 1 hour, preferably 30 minutes.

The pharmaceutically active agent is preferably a drug which may suitably be delivered by inhalation to a target subject. An exemplary but non-limiting list includes, for example, steroids, hormones, therapeutic proteins and peptides, beta-2 agonists, bronchodilators, corticosteroids and antihistamines. In particular, the drug is preferably selected from salbutamol, terbutaline, insulin, calcitonin, human growth hormone, cromolyns, beclomethasone, budesonide, mometasone, ciclesonide, triamcinolone, fluticasone, rofleponide, salmeterol, formoterol and pharmaceutically acceptable salts, hydrates and solvates thereof, which may be deposited on the carrier particle surface and subsequently delivered to a subject.

The surface properties produced by the process disclosed above are important as they are believed to improve the deposition profile of the pharmaceutically active agent. Thus, a lower quantity of drug may be deposited on the carrier particle, and a greater proportion of the drug per unit weight of deposited drug may be delivered to the subject.

It has been found that the presence of a partially amorphous surface resulted in improved blending of salbutamol sulphate with the carrier (coefficient of variation of 3.0 compared with 4.5 or more with any combination of crystalline lactose samples studied).

### EXAMPLE 1

Amorphous lactose fines were prepared by spray drying to give fines which are approximately 100% amorphous and approximately less than 10 μm in size. The amorphous lactose fines were produced by spray drying a solution of lactose monohydrate (Borculo Domo Ingredients). A 10% w/w solution was prepared by dissolving 50.0 g of lactose in purified water (approximately 400 mL). The solution was stirred in a 500 mL beaker using a magnetic stirrer accompanied by gentle heating. The volume of the solution was then made up to 500 mL with purified water and used in the mini spray dryer (Buchi 190). The parameters used are outlined in Chidavaenzi et al (The Use of Thermal Techniques to Assess the Impact of Feed Concentration on the Amorphous Content Polymorphic Forms Present in Spray Dried Lactose, Int. J. Pharmaceutics, 159, 57-74, 1997). Confirmation that the fines were 100% amorphous was obtained by assessing the powder in an isothermal micro-calorimeter at 25° C. by exposing the powder to 75% relative humidity as described by Darcy et al (Quantitative Assessments of Powder Crystallinity: Estimates Of Heat and Mass Transfer to Interpret Isothermal Microcalorimetry Data, Thermochemica acta, 316, 29-36 1998).

Coarse carrier crystals of lactose (5.01078 g, Pharmatose 325M, DMV international) were airjet sieved (Alpine, Germany) for 15 minutes over a 45 micron mesh to remove any fines in the formulation. This core was then blended with 10% w/w amorphous fines (0.50672 g); The overall percentage of amorphous fines in final formulation was 8.7 % w/w.

The two powders may be mixed in a Turbula mixer in two incremental steps (total mixing time 10 minutes, 90 rpm), or via a single step in a Turbula mixer, 42 rpm, 30 minutes.

The powder blend was then placed in a plastic tube. The mix is held in place by two plastic discs covered in filter paper. The discs have small holes for air to pass through. Air is pumped through the apparatus at 0.2 L/min. At first it passes though a saturated solution of magnesium nitrate before passing through the powder. This raises the relative humidity to 53% RH.

The humidifying time (crystallisation) was varied to include 0.5, 1, 2, 3, 4 and 5 hours. The powders were then dried for 24 hours. Sieving over a 90 μm mesh removed any large aggregates from the powder. Scanning electron micrographs of the particles were produced on a Philips Model SEM XL20 (Philips Electronics, Eindhoven, Netherlands) and the amorphous content of the crystallised lactose determined.

The amorphous content of the carrier particle is determined using a Thermometric 2225 precision solution calorimetry as described in Hogan et al, (Int. J. Pharmaceutics, 207, 57-64, 2000).

This conditioned carrier was then blended with micronised salbutamol sulphate. The mixing took place in a Turbula mixer (30 minutes, 42 rpm).

The uniformity of drug content was then analysed by removing 10 samples from the mix. Each sample (10 mg) was dissolved in 0.1M HCl and the drug content analysed by UV at 276 nm. From this the % w/v is calculated using a calibration curve already set up for salbutamol. The exact % w/w of drug in the sample is then calculated. The mean, standard deviation and coefficient of variation (which equals standard deviation divided by the mean multiplied by 100) are then reported.

The coefficient of variation is an indication of how uniformly the drug is distributed in the mix. The lower the figure the better. Only blends with coefficient of variation less than 5% are used to produce a product.

The powder is then packed into the reservoir dry powder inhaler (the Clickhaler, Innovata Biomed).

The respirable fraction of salbutamol sulphate is assessed by actuating the inhaler into a twin stage impinger set at flow rate 60 L/min. The inhaler is actuated ten times per run. The respirable fraction is quantified by UV. This is a standard apparatus used routinely in quality control (European Pharmacopoeia).

The amount of drug recovered from both stages 1 and 2 is the emitted dose. The amount in stage 2 is the respirable dose and the fine particle fraction (FPF) is the respirable fraction over the emitted dose multiplied by a hundred. The higher the fine particle fraction the better because more of the drug is respirable. Impinger runs were carried out on the different product derived from the differing lengths of conditioning. The results are shown in Table 1. A number of impinger runs were carried out for each carrier particle product.

It is clear from table 1, that there is an optimum amorphous content of the carrier. This appears to be influenced by the process conditions and the amount of fines initially added to the fines/core mix. In particular, 10% added fines and conditioning for 2 hours produces the best results.

### COMPARATIVE EXAMPLE 1

Lactose monohydrate was sieved to 63-90 microns size range. It was milled in a ball mill using ceramic balls for 30 minutes at high speed, 60 rpm, following the general method set out in WO01/05429. In fact, the amorphous content produced was 1.5%. Also, a great reduction in particle size was seen by Scanning Electron Micrographs and by particle sizing method.

### COMPARATIVE EXAMPLE 2

Lactose monohydrate was sieved to 63-90 microns size range. It was milled in a ball mill using light plastic balls for 6 hours at a speed of 30 rpm, following the general method set out in WO01/05429. The amorphous content produced was 1.6%. Particle size was reduced but not the same extent as in Comparative Example 1.

This concludes that milling does not result in an amorphous content as high as that required for the carrier of the present invention.

### COMPARATIVE EXAMPLE 3

Lactose monohydrate was air jet sieved to remove the fines. 10% crystalline fines were then added and the blend conditioned at 53% RH for 2 hours then dried. The amorphous content of the carrier was 0.9%. The carrier was blended with 4% w/w salbutamol sulphate. Analysis in the twin impinger showed that the average fine particle fraction of drug liberated was 22.5%. This shows that the amorphous content as well as the conditioning step are necessary in achieving good drug delivery performance. The results are shown in Table 2.

### COMPARATIVE EXAMPLE 4

Lactose monohydrate was air jet sieved to remove the fines. 15% (in example 1, 10% fines were added) amorphous fines were then added and the blend conditioned at 53% RH for 2 hours then dried. The average amorphous content of the carrier was 6.8%. The carrier was blended with 4% w/w salbutamol sulphate. Analysis in the twin impinger showed that the average fine particle fraction of drug liberated was 19%. The results are shown in Table 2. The 15% addition of fines results in fines aggregating together away from the carrier and this can be seen by Scanning. Electron Microscopy. This results in a high amorphous content due to the aggregated fines. This example illustrates the requirement for a core particle with an amorphous surface.

### COMPARATIVE EXAMPLE 5

A crystalline carrier without fine particles provided a product whose analysis in the twin impinger showed that the average fine particle fraction of drug liberated was 25%

TABLE 1
Salbutamol sulphate performance in the Twin Impinger.

| | Emitted | Respirable | Fine particle |
|---|---|---|---|
| Carrier lactose | dose (mg) | dose (mg) | fraction (%) |
| 10% added amorphous fines, | 1.32 | 0.29 | 22 |
| 0 hr conditioned (Drying | 1.43 | 0.37 | 25.9 |
| step only) | 1.65 | 0.4 | 24.2 |
| 10% added amorphous fines, | 0.83 | 0.13 | 15.7 |
| 0.5 hr conditioned | 0.98 | 0.13 | 13.3 |
| 10% added amorphous fines, | 1.14 | 0.28 | 24.6 |
| 1 hr conditioned | 1.17 | 0.33 | 28.2 |
| | 1.12 | 0.28 | 25 |
| 10% added amorphous fines, | 0.98 | 0.42 | 42.9 |
| 2 hr conditioned | 1 | 0.42 | 42 |
| | 1.13 | 0.42 | 37.2 |
| | 1.04 | 0.36 | 34.6 |
| | 1.14 | 0.43 | 37.7 |
| | 1.4 | 0.64 | 45.7 |
| | 0.94 | 0.34 | 36.1 |
| | 1.75 | 0.78 | 44.5 |
| | 1.83 | 0.78 | 42.6 |
| | 1.31 | 0.4 | 30.1 |
| 10% added amorphous fines, | 0.97 | 0.29 | 29.9 |
| 2 hr conditioned. Drug broken up | | | |
| by spatula rather than mesh | 0.97 | 0.34 | 35.1 |
| sieve | | | |
| 10% added amorphous fines, | 0.79 | 0.16 | 20.3 |
| 3 hr conditioned | 0.92 | 0.17 | 18.5 |
| | 1.06 | 0.25 | 23.6 |
| 10% added amorphous fines, | 0.96 | 0.12 | 12.5 |
| 4 hr conditioned | 1.03 | 0.22 | 21.4 |
| | 1 | 0.23 | 23 |
| Addition of 10% amorphous | 0.94 | 0.23 | 24.5 |
| fines (no conditioning, no | 1.03 | 0.2 | 19.4 |
| drying) | 1.08 | 0.29 | 26.9 |
| | 1.03 | 0.28 | 27.2 |

TABLE 2
Comparative Examples (using Salbutamol sulphate)
performance in the Twin Impinger.

| | Emitted | Respirable | Fine particle |
|---|---|---|---|
| Carrier lactose | dose (mg) | dose (mg) | fraction (%) |
| Addition of 10% crystalline fines | 2.01 | 0.28 | 13.9 |
| and 2 hr conditioning | 0.89 | 0.23 | 25.8 |
| | 1.11 | 0.31 | 27.9 |
| Addition of 15% amorphous | 0.86 | 0.12 | 14 |
| fines and 2 hr conditioning | 1.09 | 0.22 | 20.2 |
| | 1.35 | 0.31 | 23 |

TABLE 3
Final carrier amorphous content for 100% amorphous added fines.

| | Amorphous content |
|---|---|
| Carrier | % (Means) |
| 10% added fines, 0 hr conditioned | 10.2 |
| 10% added fines, 0.5 hr conditioned | 8.2 |
| 10% added fines, 1 hr conditioned | 7.3 |
| 10% added fines, 2 hr conditioned | 6 |
| 10% added fines, 3 hr conditioned | 4.4 |
| 10% added fines, 4 hr conditioned | 1.9 |
| 10% added fines, 5 hr conditioned | 1.1 |
| +10% crystalline fines, 2 hr conditioned | 0.9 |
| +15% amorphous fines, 2 hr conditioned | 6.8 |

1. A particulate substrate suitable for carrying a drug for delivery, comprising a substantially crystalline core and a surface coating, wherein the particulate substrate has a proportion of amorphous character of 2% or greater by weight of particulate substrate.

2. A substrate according to claim 1, the surface coating has a proportion of amorphous character such that the total amorphous content of the carrier particle is in the range of 2 to 80%, more preferably 3 to 20%, more preferably 3.5 to 8%, most preferably about 4 to 7% by weight of the carrier particle.

3. A substrate according to claim 1 or 2, having a diameter in the range of 10 μm to 500 μm, preferably 20 μm to 100 μm, most preferably 45 μm to 90 μm.

4. A substrate according to claim 1, 2 or 3, wherein the core material is selected from saccharides, preferably lactose, sucrose, glucose, galactose, fructose, trehalose, raffinose and mixtures thereof, most preferably lactose.

5. A substrate according to any preceding claim, wherein the surface material comprises saccharides, preferably lactose, sucrose, glucose, galactose, fructose, trehalose, raffinose and mixtures thereof, most preferably lactose.

6. A substrate according to any preceding claim wherein the surface of the substrate is formed from the same material as the core.

7. A substrate according to any preceding claim additionally comprising a pharmaceutically active agent substantially at the surface, preferably a drug, most preferably a drug which may be delivered by inhalation.

8. A substrate according to claim 7, wherein the drug is selected from steroids, hormones, therapeutic proteins and peptides, beta-2 agonists, bronchodilators, corticosteroids and antihistamines.

9. A substrate according to claim 7 or 8, wherein the drug is selected from the group consisting of salbutamol, terbutaline, insulin, calcitonin, human growth hormone, cromolyns, beclomethasone, budesonide, mometasone, ciclesonide, triamcinolone, fluticasone, rofleponide, salmeterol, formoterol and pharmaceutically acceptable salts, hydrates and solvates thereof.

10. process for the production of a particulate substrate comprising the steps of:
a) mixing substantially crystalline particles having an average diameter greater than 10 μm with at least partially amorphous particles having average diameters less than 10 μm;
b) exposing the mixture to conditions capable of inducing crystallization of the amorphous particles for a predetermined period in order that partial crystallization takes place.

11. A process according to claim 10, wherein the crystalline particle is selected from saccharides, preferably lactose, sucrose, glucose, galactose, fructose and mixtures thereof, most preferably lactose.

12. A process according to claim 10 or 11, wherein the crystalline particles have a diameter in the range of 10 μm to 500 μm, preferably 20 μm to 100 μm, most preferably 40 μm to 95 μm.

13. A process according to any of claims 10 to 12, wherein the at least partially amorphous particles have a diameter in the range of about 0.1 μm to about 10 μm, preferably about 1 μm to about 8 μm, most preferably a diameter of about 5 μm.

14. A process according to any of claims 10 to 13, wherein the at least partially amorphous particles are produced by a process selected from spray drying, freeze drying and precipitation.

15. A process according to any of claims 10 to 14, wherein the amorphous particles, prior to the crystallisation process, have an amorphous character of greater than 10%, preferably greater than 50%, most preferably between 90 and 100%.

16. A process according to any of claims 10 to 15, wherein the proportion of crystalline particles to amorphous particles in the mix is in the ratio range of from 20:1 to 5:1, preferably 12:1 to 7:1 by weight of the mixture, most preferably the coarse particles to fine particles ratio being about 9:1 by weight of the mixture.

17. A process according to any of claims 10 to 16, wherein the conditions for inducing crystallization of the amorphous particles comprise exposure to humidity, preferably a relative humidity in the range of 1 to 100%, more preferably 30% to 80%, most preferably 40 to 60% relative humidity.

18. A process according to any of claims 10 to 17, wherein exposure to crystallization inducing conditions is for a period in the range of 10 seconds to 48 hours, preferably 10 minutes to 6 hours, more preferably 30 minutes to 5 hours, most preferably 1 hour to 3 hours.

19. A process according to any of claims 10 to 18, wherein exposure to crystallization inducing conditions comprises exposure to an elevated temperature, preferably a temperature in the range of 5° C. to 200° C., more preferably 10° C. to 80° C., most preferably 15° C. to 50° C.

20. A process according to any of claim 10 to 19, comprising the additional step of drying the particles.

21. A process according to any of claims 10 to 20, comprising the additional step of mixing a pharmaceutically active agent with the particulate substrate, mixing preferably taking place after step (b).

22. A process according to claim 21, wherein the pharmaceutically active agent is a drug, preferably selected from the group consisting of A substrate according to claim 7, wherein the drug is selected from steroids, hormones, therapeutic proteins and peptides, beta-2 agonists, bronchodilators, corticosteroids and antihistamines.

23. A process according to claim 22, wherein the drug is selected from the group consisting of salbutamol, terbutaline, insulin, calcitonin, human growth hormone, cromolyns, beclomethasone, budesonide, mometasone, ciclesonide, triamcinolone, fluticasone, rofleponide, salmeterol, formoterol and pharmaceutically acceptable salts, hydrates and solvates thereof.

24. A process according to claim 22, wherein the drug is in a micronised form.

25. A method for drug delivery, comprising presenting a particulate substrate having any of the features of claims 7, 8 or 9, in a dispenser and delivering the particulate substrate by inhalation to a human or animal body

26. A hermetically sealed pack comprising a particulate substrate according to any of claims 7, 8 or 9.
